(19) United States
(12) Patent Application Publication
(10) Pub. No.: US 2026/0265726 A1
(43) Pub. Date: Sep. 10, 2026

(54) SYSTEMS AND METHODS FOR ISOLATING HIGH MOLECULAR WEIGHT DNA FROM PROKARYOTIC CELLS IN STOOL

(71) Applicant: Exir, Inc., Sunnyvale, CA (US)

(72) Inventor: Morteza Roodgar, Menllo Park, CA (US)

(73) Assignee: Exir, Inc., Sunnyvale, CA (US)

(21) Appl. No.: 19/556,266

(22) Filed: Mar. 4, 2026

Related U.S. Application Data
(60) Provisional application No. 63/766,566, filed on Mar. 4, 2025.

(51) Int. Cl. C12N 15/10

(52) U.S. Cl. CPC C12N 15/1003

(57) ABSTRACT

Extracting high molecular weight DNA from prokaryotic cells in stool samples is achieved through a process involving preparation in a prechilled buffer solution, homogenization, and treatment with lytic enzymes and a chaotropic agent. The process includes mechanical disruption and application of an adsorbent for contaminant removal. A buffer solution with a pH-stabilizing agent and an ionic strength modifier is used, along with mechanical homogenization tools and lytic enzymes such as lysozyme, mutanolysin, and lysostaphin. A chaotropic agent and an adsorbent are also utilized to facilitate DNA extraction. This process enables the extraction of high-quality DNA from stool samples for various applications, including genomic analyses and diagnostic testing.

### CROSS-REFERENCE TO RELATED APPLICATIONS

This application claims the priority benefit of U.S. Provisional Patent Application Ser. No. 63/766,566, filed Mar. 4, 2025, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of biotechnological methods for DNA extraction, and more specifically to the field of high molecular weight DNA isolation from prokaryotic cells in stool samples.

### BACKGROUND

In the field of biotechnology, particularly in the area of genomics, the analysis of genetic material is used for a variety of applications, from medical diagnostics to environmental monitoring. The foundational step in such analyses involves the extraction of DNA from biological samples. The complexity of these samples can vary depending on their source, which may range from simple cultured cells to complex tissue samples or environmental collections.

### SUMMARY

In some aspects, the techniques described herein relate to a method for extracting high molecular weight DNA from prokaryotic cells in stool samples, the method including: receiving a biological sample including prokaryotes; preparing the biological sample in a prechilled buffer solution; homogenizing the biological sample through a first mechanical means for partial lysis; partially disrupting prokaryotic cell walls by treating the biological sample with at least one lytic enzyme to release genetic information; treating the biological sample with a chaotropic agent; disrupting the biological sample by a second mechanical means; and applying an adsorbent to the biological sample.

In some aspects, the techniques described herein relate to a reagent system for extracting high molecular weight DNA from a stool sample including one or more prokaryotic cells, the reagent system including: a buffer solution for preparing a biological sample, wherein the buffer solution includes a pH-stabilizing agent and an ionic strength modifier; at least one mechanical homogenization tool configured for bead beating with one or more beads; at least one lytic enzyme for disrupting prokaryotic cell walls, the at least one lytic enzyme including lysozyme, mutanolysin, lysostaphin, or a combination thereof; a chaotropic agent configured for treating the biological sample; and an adsorbent configured for contaminant removal.

In some aspects, the techniques described herein relate to a kit for extracting high molecular weight DNA from stool samples including prokaryotic cells, the kit including: a first container including a buffer solution including a pH-stabilizing agent and an ionic strength modifier; one or more beads configured for mechanical homogenization through bead beating, wherein the one or more beads include glass or ceramic; at least one receptacle including at least one lytic enzyme selected from a list consisting of: lysozyme, mutanolysin, lysostaphin, or a combination thereof; a second container including a chaotropic agent; and a third container including an adsorbent configured for binding and removing one or more contaminants from a stool sample.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates an embodiment of a method for extracting high molecular weight DNA from prokaryotic cells in a sample.

FIG. 2A illustrates an embodiment of a reagent system used for extracting high molecular weight DNA from a sample.

FIG. 2B shows a block diagram illustrating the reagent system from FIG. 2A, according to some embodiments.

FIG. 3A illustrates an embodiment of a kit for extracting high molecular weight DNA from a stool sample.

FIG. 3B shows a block diagram illustrating the kit from FIG. 3A, according to some embodiments.

FIG. 3C shows a block diagram illustrating the kit from FIG. 3A, according to some embodiments.

FIG. 4 shows experimental results of initial high molecular weight DNA extraction with agarose gel electrophoresis.

FIG. 5 shows experimental results of high molecular weight DNA after phenol/chloroform clean-up with agarose gel electrophoresis.

FIG. 6 shows experimental results of high molecular weight DNA after RNAse purification with agarose gel electrophoresis.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, is necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure. Disclosed herein are systems and methods for a method for extracting high molecular weight DNA from prokaryotic cells in a stool sample. The stool sample may be from a mammal, a human, an animal, and the like.

A technological problem with the extraction of high molecular weight DNA from biological samples is the process of centrifugation. Centrifugation is commonly used to separate cellular components and concentrate DNA from lysed cells. However, this process can exert substantial shear forces on the DNA molecules, particularly when dealing with high-speed centrifugation protocols. These shear forces can cause fragmentation of the DNA, leading to shorter DNA fragments that are less useful for long-read sequencing technologies. This issue is exacerbated in samples containing a high density of cellular debris and other particulate matter, which are common in complex biological samples such as stool.

In the context of prokaryotic cells, particularly bacteria found in stool samples, the technical problem is even more pronounced due to the nature of these cells. Prokaryotic cells, unlike eukaryotic cells, do not have a nucleus to protect their genetic material. As a result, the DNA in prokaryotic cells is more susceptible to mechanical stresses during the extraction process. When these cells undergo mechanical lysis, the DNA is often exposed to shearing forces, which can break the DNA into smaller fragments. This fragmentation is particularly problematic for applications utilizing high molecular weight DNA, as it compromises the ability to perform detailed genomic analyses.

The shearing of DNA during centrifugation and other mechanical stresses is a technical problem that limits the effectiveness of existing DNA extraction methods for non-nucleated cells. Traditional mechanical lysis techniques, such as bead beating or vortexing, combined with centrifugation, often result in DNA fragmentation. This technical problem is further compounded by the need to balance effective cell disruption with the preservation of DNA integrity. Maintaining the DNA in an intact, high molecular weight form supports subsequent sequencing and analysis that rely on long, contiguous DNA strands to provide comprehensive genomic information.

The technical problems associated with extracting high molecular weight DNA from non-nucleated cells, such as those encountered in stool samples, may hinder current research and/or diagnostic efforts. The inability to consistently obtain intact DNA fragments impairs the effectiveness of long-read sequencing technologies, which are used for detailed genomic analyses. This limitation affects various applications, including the accurate identification of microbial species and subspecies, the study of microbial diversity, and/or the exploration of complex microbial interactions within the gut ecosystem. Furthermore, the compromised DNA integrity reduces the reliability of biomarker discovery and disease association studies, ultimately affecting the development of targeted therapies and/or personalized medicine approaches. Addressing these technical problems advances the fields of microbial genomics, medical diagnostics, and/or therapeutic development, enabling more precise and/or comprehensive genomic studies that can drive innovation and improve health outcomes.

To address these technical problems, described herein are technological solutions, optimized DNA extraction methods that may lyse non-nucleated cells and separate DNA while reducing shearing. The methods described herein solve the above technical problems with technical solutions by increasing and/or improving the yield and quality of high molecular weight DNA, facilitating more accurate and/or detailed genomic studies. The methods described herein solve the above technical problems with technical solutions by minimizing DNA fragmentation during centrifugation and mechanical lysis, which would represent an advancement in the field of microbial genomics and related applications.

Given some challenges associated with extracting high molecular weight DNA from non-nucleated cells, particularly in the context of stool samples, there is a clear need for improved methods that can overcome these obstacles. Current techniques, while useful in some scenarios, often fall short in maintaining the integrity of the DNA due to the harsh mechanical and/or centrifugal forces involved. This limitation hinders the ability to perform advanced genomic analyses, which typically implement long, intact DNA molecules to provide comprehensive and/or accurate results.

The methods described herein for extracting high molecular weight DNA from stool samples solve the above technical problems with technological solutions by effectively balancing the need for thorough cell lysis with the preservation of DNA integrity. This includes minimizing the shear forces exerted during mechanical disruption and centrifugation. Additionally, the methods may be capable of separating DNA from complex mixtures of cellular debris and other contaminants typically found in stool samples. The methods described herein solve the above technical problems with technical solutions to facilitate the extraction of high-quality DNA suitable for long-read sequencing and/or other downstream applications.

### Practical Applications

The methods described herein provide applications for a wide range of fields, including medical diagnostics, therapeutic development, and microbial genomics. In medical diagnostics, for example, a practical application of the methods described herein includes analyzing high-quality microbial DNA from stool samples to identify (e.g., more accurately) microbial profiles associated with various diseases. This, in turn, could facilitate the development of targeted therapies and personalized medicine approaches. In microbial genomics, a practical application of the methods described herein includes determining microbial diversity and/or function, contributing to research in areas such as gut microbiome studies and environmental microbiology.

The practical applications of the methods and compositions described herein include improved medical diagnostics; therapeutic developments; medical genomics; personalized medicine; and environmental monitoring. Various practical applications of the methods and compositions may be further described elsewhere herein. The practical applications mentioned are intended as various examples, and the express mention of some examples is not meant to be limiting. Practical applications not mentioned may practice the methods and compositions described herein.

A practical application of the described methods is in the field of medical diagnostics. The ability to extract high molecular weight DNA from stool samples allows for comprehensive microbial profiling through advanced sequencing techniques. For instance, in patients with gastrointestinal disorders such as inflammatory bowel disease (IBD) or colorectal cancer, this method can identify specific microbial signatures associated with these conditions. The extracted DNA can be sequenced to provide a detailed analysis of the gut microbiome, revealing pathogenic bacteria that may be contributing to the disease. This detailed microbial profile can lead to early diagnosis and the development of personalized treatment plans.

The methods described herein may also be practically applied for the development of new therapies. By extracting high-quality DNA from stool samples, the composition and/or function of the gut microbiome may be determined. This information may be used for developing targeted probiotics, prebiotics, and other therapeutic interventions aimed at restoring a healthy gut microbiome. Such treatments can be particularly beneficial for conditions like obesity, diabetes, and mental health disorders where the gut microbiome contributes to outcome, disease course, and/or disease severity.

In microbial genomics research, a practical application of the methods described herein includes enabling sequencing of microbial genomes from stool samples. Sequencing of microbial genomes may be used for analyzing microbial diversity, evolution, and/or function. The high molecular weight DNA obtained allows for long-read sequencing, providing more accurate and complete genome assemblies.

A further application of the methods described herein includes personalized microbiome profiling, which can be used to tailor medical treatments and dietary recommendations to individual patients. By analyzing the microbial composition of a patient's gut, personalized interventions can be developed or recommended that address specific health issues. This personalized approach is particularly useful for managing chronic diseases, optimizing digestive health, and enhancing overall well-being.

A still further application of the methods described herein includes environmental monitoring by analyzing the gut microbiomes of wildlife or livestock to determine or assess the impact of environmental changes, pollutants, and other factors on wildlife health.

### Materials, Methods, and Compositions

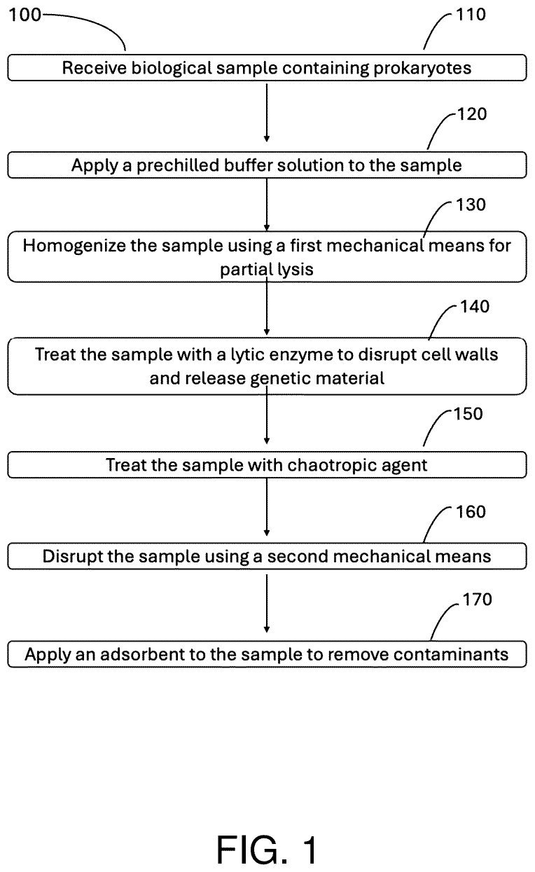

As shown in FIG. 1, an embodiment of a method 100 for extracting high molecular weight DNA from prokaryotic cells in a stool sample includes receiving the sample at block 110. Stool samples including prokaryotic cells are collected at block 110 from a source, for example, a human, nonhuman primate, mouse, dog, cat, and the like. The samples may be collected using sterile or aseptic techniques to reduce or prevent outside contamination and stored at low temperatures (e.g., about 1° C. to about 10° C.; about 2° C. to about 5° C.; about 3° C. to about 4° C.) to preserve DNA integrity until processing.

An embodiment of a method 100 for extracting high molecular weight DNA from prokaryotic cells in a stool sample includes applying a prechilled buffer solution to the sample at block 120. The buffer solution may include a pH-stabilizing agent, an ionic strength modifier, and optionally a nuclease inhibitor or chelating agent. The pH-stabilizing agent may include, but is not limited to, Tris-HCl, 4-(2-hydroxyethyl)-1-piperazineethanesulfonic acid (HEPES), a phosphate buffer, 3-(N-morpholino)propanesulfonic acid (MOPS), and the like. For example, the pH-stabilizing agent may have a concentration of about 10 mM to about 50 mM; about 10 mM to about 80 mM; about 20 mM to about 55 mM; about 30 mM to about 55 mM; about 40 mM to about 55 mM; about 40 mM to about 60 mM; about 45 mM to about 55 mM; about 50 mM, etc. The ionic strength modifier may include, but is not limited to, sodium chloride (NaCl), potassium acetate, and sodium acetate. The ionic strength modifier may have a concentration of about 50 mM to about 150 mM. The optional nuclease inhibitor or chelating agent helps maintain the integrity of the DNA during extraction. The optional nuclease inhibitor may include, but is not limited to, ethylenediaminetetraacetic acid (EDTA). The concentration of the optional nuclease inhibitor or chelating agent may be about 0.5 mM to about 5 mM; about 0.5 mM to about 2.5 mM; about 0.5 mM to about 1.5 mM; or about 1 mM, etc. The application of the prechilled buffer solution at block 120 to the sample collected at block 110 may increase or maintain the integrity of the DNA by providing a tuned environment for subsequent enzymatic reactions.

An embodiment of a method 100 for extracting high molecular weight DNA from prokaryotic cells in a stool sample includes homogenizing the sample using a first mechanical means for partial lysis. Homogenization may be used to at least partially disrupt the walls of cells in the sample. Homogenizing may include vigorously mixing, vortexing, bead beating, and the like. For example, the sample may be vigorously mixed or vortexed for about 0.5 minutes to about 5 minutes; about 0.5 minutes to about 2 minutes; about 0.5 minutes to about 1.5 minutes; about 1 minute, etc. In some embodiments where bead beating is used for homogenization, the bead may include, be formed of, or comprise one or more of: glass, ceramic, silica, zirconia, a combination thereof, and the like. A diameter of the beads may be about from about 0.05 mm to about 5 mm; about 0.1 mm to about 1 mm; about 0.1 mm to about 0.5 mm; about 0.1 mm to about 3 mm; about 0.1 mm; about 0.5 mm; about 2.8 mm; about 3.0 mm, etc. Mechanical lysis or homogenization may help to break down the cell walls and release some of the cellular contents in the sample. Following the initial homogenization, the sample is centrifuged at about 12,000 rpm to about 15,000 rpm for about 2 minutes to about 5 minutes. The supernatant is then transferred to a clean microcentrifuge tube.

An embodiment of a method 100 for extracting high molecular weight DNA from prokaryotic cells in a stool sample includes treating the sample with a lytic enzyme to disrupt cell walls and release genetic material at block 140. In some embodiments, block 140 may be performed before or after block 130. The lytic enzymes may include lysozyme, mutanolysin, lysostaphin, a combination thereof, and the like. For example, lysozyme may be used to break down or digest bacterial cell walls. The lysozyme may be used at a concentration of about 200 mg/mL to about 300 mg/mL; about 200 mg/mL to about 250 mg/mL; about 250 mg/mL to about 300 mg/mL; about 225 mg/mL to about 275 mg/mL; about 220 mg/mL to about 260 mg/mL; about 240 mg/mL to about 260 mg/mL; etc. For instance, mutanolysin may be used to target bacterial cell wall components. The mutanolysin may be used at a concentration of about 20 KU/mL to about 30 KU/mL. For example, lysostaphin may be used to break down or digest the cell walls. Lysostaphin may be used at a concentration of about 3 KU/mL to about 5 KU/mL, about 1 KU/mL to about 10 KU/mL; about 2 KU/mL to about 7 KU/mL; etc. The method may further include incubating the sample with one or more enzymes at a predefined temperature or in a predefined temperature range for a predefined duration of time or range of time to ensure thorough cell lysis. For example, the predefined temperature may be about 35° C. to about 42° C. or about 37° C. The predefined range of time may be about 15 minutes to about 90 minutes; about 30 minutes to about 60 minutes; about 20 minutes to about 40 minutes; about 40 minutes to about 70 minutes; etc. Block 140 functions to enhance enzyme activity and/or ensure effective breakdown of the cell walls in the sample.

An embodiment of a method 100 for extracting high molecular weight DNA from prokaryotic cells in a stool sample includes treating the sample with a chaotropic agent at block 150. The chaotropic agent may be used to denature proteins and other contaminants to facilitate the isolation of the DNA. In some non-limiting embodiments, the chaotropic agent may include guanidinium thiocyanate, and the like. The chaotropic agent may be used at a concentration of about 3 M to about 7 M; about 1 M to about 10 M; about 2 M to about 8 M; about 1 M to about 8 M; etc. The method may further include incubating the sample with one or more chaotropic agents and mixing gently for a predefined duration of time. The predefined duration of time may be about 30 seconds to about 60 seconds. The method may further include adding a detergent followed by mixing well and performing centrifugation. In some non-limiting embodiments, the detergent may be about 8% to about 12% N-Laurylsarcosine. The sample may then be incubated at about 35° C. to about 42° C. for about 5 minutes to about 15 minutes with mixing at about 250 rpm to about 350 rpm, followed by an incubation at about 65° C. to about 75° C. for about 45 minutes to about 75 minutes. The method may further include adjusting the concentration of one or more chaotropic agents based on the density of prokaryotic cells in the sample. The chaotropic agent may help to solubilize cellular components and/or protect the DNA from shearing.

In some embodiments, the method may further include assessing the sample for the presence or absence of one or more enzymatic inhibitors. For example, the assessment may be performed using mass spectrometry, spectrophotometry, and the like. The sample may be assessed to determine whether enzymatic inhibitors are present that could interfere with the DNA extraction process. Alternatively, or additionally, the assessment may be used to ensure the integrity and/or quality of the DNA extraction product. In some embodiments, the method further includes performing one or more genomic analyses on the purified DNA.

An embodiment of a method 100 for extracting high molecular weight DNA from prokaryotic cells in a stool sample includes disrupting the sample using a second mechanical means at block 160. The sample may be disrupted by a second mechanical means to increase disruption of any remaining cell structures. The second mechanical means may include bead beating, vortexing, ultrasonic homogenization, or high-speed blending. For example, the sample may be subjected to bead beating with cycles of about 20 to about 40 seconds of beating followed by 1.5 to 3 minutes of rest. In some embodiments where bead beating is used for homogenization, the bead may include, be formed of, or comprise one or more of: glass, ceramic, silica, zirconia, a combination thereof, and the like. A diameter of the beads may be about from about 0.05 mm to about 5 mm; about 0.1 mm to about 1 mm; about 0.1 mm to about 0.5 mm; about 0.1 mm to about 3 mm; about 0.1 mm; about 0.5 mm; about 2.8 mm; about 3.0 mm; etc. The disruption at block 160 may include a cycle, one or more cycles of disruption, or a plurality of cycles. For example, a cycle may include about 20 to 40 seconds of bead beating followed by about 1.5 to about 3 minutes of rest.

An embodiment of a method 100 for extracting high molecular weight DNA from prokaryotic cells in a stool sample includes applying an adsorbent to the treated mixture to reduce or remove contaminants in the sample at block 170. For example, the adsorbent may include, but is not limited to, Polyvinylpolypyrrolidone (PVPP) and the like. About 10 mg to about 20 mg of PVPP may be added to the mixture, which binds to and removes contaminants such as humic substances and polysaccharides. The method may further include mixing the sample well and performing centrifugation.

In some embodiments, the method may further include centrifuging the sample to facilitate the separation of PVPP-bound contaminants from the nucleic acids. The centrifugation may be performed at specific speeds and/or durations to reduce DNA shearing. For example, the centrifugation speed may be about 15,000×g to about 25,000×g for a duration of about 2 to about 5 minutes. After centrifugation, the supernatant containing the high molecular weight DNA may be carefully transferred to a new 2-mL centrifuge tube for further processing or analysis. The method may further include assessing the concentration and purity of the extracted DNA using a nanodrop spectrophotometer.

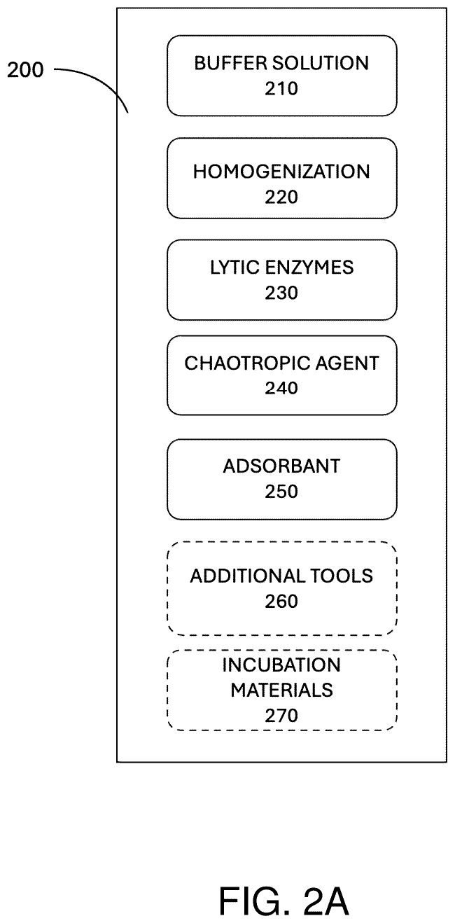

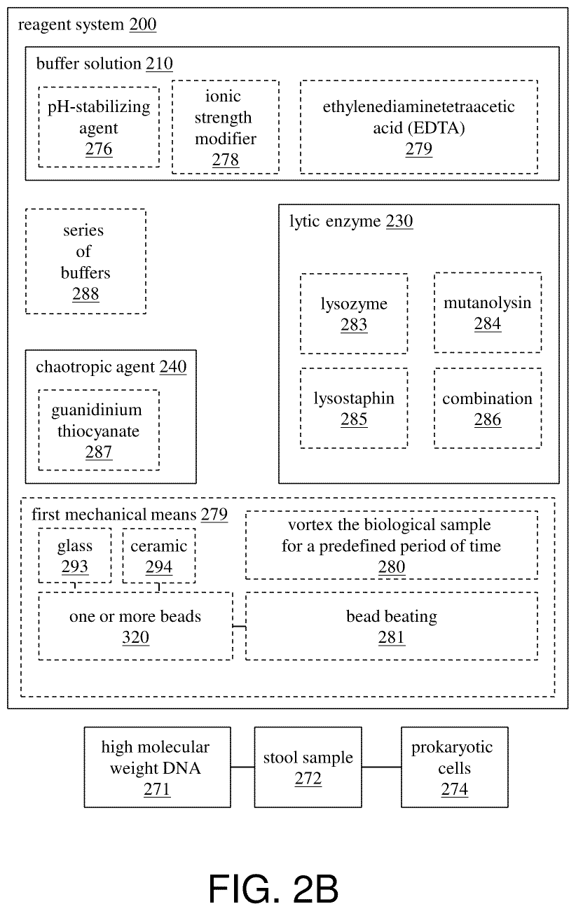

As illustrated in FIG. 2A, a reagent system 200 for extracting high molecular weight DNA from stool samples may include the following components: a buffer solution 210 for preparing the biological sample, mechanical homogenization tools 220 (e.g., bead beating means as described above, vortexing means as described above, etc.), at least one of the lytic enzymes 230 (e.g., lysozyme, mutanolysin, lysostaphin), a chaotropic agent 240 (e.g., guanidinium thiocyanate), and an adsorbent 250 for contaminant removal.

In some embodiments, the buffer solution 210 may include a pH stabilizing agent and an ionic strength modifier, as described elsewhere herein. The buffer solution 210 may optionally further include a chelating agent. For example, the pH stabilizing agent may include Tris-HCl at a concentration of about 10 mM to about 50 mM. Components such as EDTA may be added to the buffer solution 210 at a concentration of about 0.5 mM to about 1 mM to inhibit nuclease activity, as described above. Various mechanical lysis methods, performed by the mechanical homogenization tools 220, can be employed, including bead beating using glass, silica, zirconia, or ceramic beads with diameters ranging from about 0.1 mm to about 0.5 mm, or vortexing.

The concentration of the chaotropic agent, such as guanidinium thiocyanate, can be adjusted based on the prokaryotic cell density in the sample. For high-density samples, a concentration of about 4 M to about 6 M may be used, while lower concentrations may be adequate for less dense samples. The incubation temperature for enzymatic treatment can be set to optimize enzyme activity. For instance, lysozyme activity is effective at about 37° C., while other enzymes may be effective at different temperatures.

In some embodiments, one or more buffers, enzymes, homogenization tools, agents, receptacles, or other components described herein are sold, commercialized, marketed, advertised, or otherwise packaged individually or bundled together as a reagent system or in a kit. A reagent system or kit may include one or more buffers, enzymes, homogenization tools, agents, receptacles for receiving one or more cells, one or more samples, one or more extracts, and/or any other component for completing all or a portion of the methods described herein.

The reagent system 200 may further include an adsorbent 250, for example, PVPP for contaminant removal. An adsorbent binds to and removes contaminants such as humic substances and polysaccharides, ensuring the purity of the extracted DNA. Using an adsorbent may improve the quality of the extracted DNA for downstream applications, such as genomic analyses and diagnostic testing. Other materials that can be used for contaminant removal may include washing the phenol at least two times: a chloroform phase, from a phenol chloroform extract, with chloroform twice, to reduce phenol contaminants from the DNA before DNA alcohol precipitation. Chloroform denatures remaining lipids and proteins and allows the proper separation of the organic and aqueous phase, protecting the DNA. Another method to remove DNA contaminants can be benzyl:isoamyl alcohol in a volumetric 9:1 ratio. Another method to remove DNA contaminants from DNA may include using silica columns. The centrifugation through the silica column may break the gDNA, so a modified centrifugation step may be used. These alternatives provide flexibility in the DNA purification process, allowing for optimization based on the nature and extent of contamination present in the sample.

The reagent system 200 may optionally include additional tools 260 for further mechanical disruption or homogenization of the sample. Tools for this step may include bead mills, vortex mixers, ultrasonic homogenizers, high-speed blenders, and/or a French press. These tools offer various methods of mechanical disruption, each being used to break down different types of biological samples, thereby enhancing the overall yield and quality of the extracted DNA. The choice of tool may be adjusted based on the specific requirements of the sample type and the desired purity of the final DNA product.

The reagent system 200 may optionally include incubation materials 270 to incubate the biological sample with one or more of the following, alone or in combination: the buffer solution 210, at least one of the lytic enzymes 230, the chaotropic agent 240, the adsorbent 250, at a controlled temperature. The optional incubation materials 270 may include a temperature-controlled incubator, a water bath, a thermal cycler, and the like.

As illustrated in FIG. 2B, an example reagent system 200 is shown for extracting high molecular weight DNA 271 from a stool sample 272 containing prokaryotic cells 274. The reagent system 200 includes a buffer solution 210 that may be used during initial sample preparation and stabilization. The buffer solution 210 may include a pH-stabilizing agent 276 and an ionic strength modifier 278. In some examples, the pH-stabilizing agent 276 may include tris (hydroxymethyl) aminomethane hydrochloride (Tris-HCl), 4-(2-hydroxyethyl)-1-piperazineethanesulfonic acid (HEPES), one or more phosphate buffers, and/or 3-(N-morpholino) propanesulfonic acid (MOPS). In some examples, the ionic strength modifier 278 may include sodium chloride, potassium acetate, or sodium acetate.

In some embodiments, the buffer solution 210 further includes ethylenediaminetetraacetic acid (EDTA). EDTA may be included to reduce nuclease activity during processing by interacting with divalent cations associated with enzymatic degradation pathways. For example, EDTA may be present in concentrations ranging from about 0.5 mM to about 5 mM during sample resuspension or early-stage handling. In some implementations, EDTA may be included in one or more buffers used prior to or during lysis.

The reagent system 200 may further include a lytic enzyme 230 for disrupting prokaryotic cell walls. The lytic enzyme 230 may include lysozyme 283, mutanolysin 284, lysostaphin 285, or a combination 286. For example, lysozyme may be used to target peptidoglycan layers in Gram-positive bacteria, mutanolysin may be used to act on specific bacterial cell wall components, and lysostaphin may be used to target staphylococcal species. In some embodiments, one or more of these enzymes are introduced following initial homogenization. The reagent system 200 may further include a chaotropic agent 240. In some embodiments, the chaotropic agent 240 includes guanidinium thiocyanate 287. Guanidinium thiocyanate 287 may be introduced after enzymatic treatment to promote protein denaturation and facilitate separation of nucleic acids from cellular material. For example, guanidinium thiocyanate may be used at concentrations ranging from about 3 M to about 7 M, depending on sample composition.

In some embodiments, the buffer solution 210 is part of a modular buffer system including a series of buffers 288. The series of buffers 288 may include two or more separate buffer compositions used sequentially or otherwise in combination. For example, a first buffer may be used during prechilled sample preparation, a second buffer may be used during enzymatic digestion, and a third buffer may be used during chemical denaturation or contaminant separation. In some embodiments, an additional buffer may be used for nucleic acid resuspension following purification.

In some embodiments, each buffer in the series of buffers 288 corresponds to a different stage of the high molecular weight DNA extraction. For example, a first stage may include sample stabilization, a second stage may include enzymatic lysis, a third stage may include chemical denaturation using the chaotropic agent 240, and a fourth stage may include contaminant binding and removal. Although the term “stage” is used here, one of skill in the art will appreciate that stages may be overlapping, combined, or operated in reverse order. The reagent system 200 may further include one or more mechanical disruption tools represented by a first mechanical means 279. The first mechanical means 279 may include vortexing the biological sample for a predefined period of time 280 and bead beating 281 using one or more beads 320. In some examples, the beads 320 may include a glass 293 material, a ceramic 294 material, and/or combinations thereof. For example, glass beads may be used for gentle homogenization, while ceramic beads may be used for increased mechanical disruption of resilient microbial cell walls.

The reagent system 200 may further include one or more materials for incubating the biological sample with the buffer solution 210 at a predefined temperature. For example, the incubation materials may include a heat block, water bath, incubator, or thermal cycling device. In some examples, incubation may occur at about 35° C. to about 42° C. during enzymatic digestion and at higher temperatures during chemical denaturation. Together, the components illustrated in FIG. 2B may be used in a staged workflow that combines chemical, enzymatic, and mechanical processing to isolate high molecular weight DNA from stool samples containing prokaryotic cells.

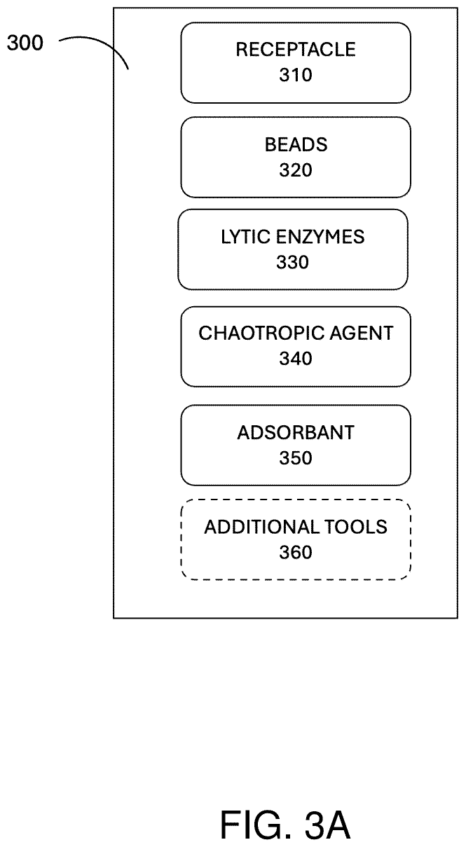

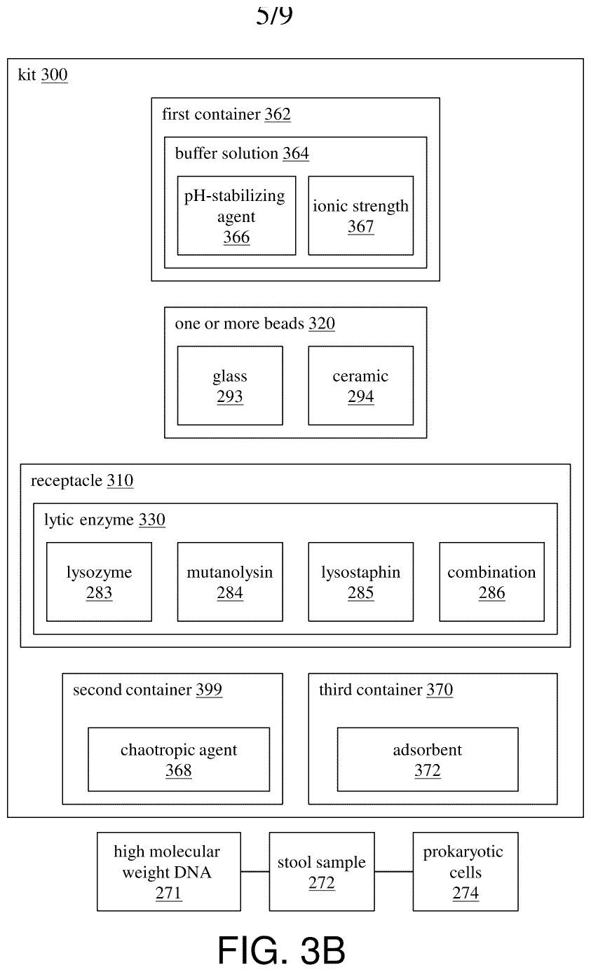

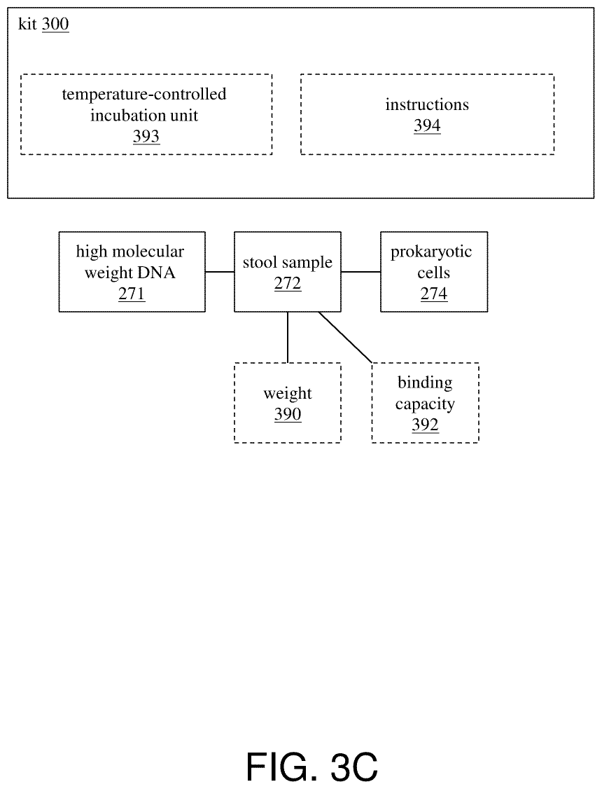

As illustrated in FIG. 3A and described elsewhere herein, a kit 300 for extracting high molecular weight DNA from stool samples comprising prokaryotic cells may include the following components: a receptacle 310 for preparing the biological sample. The receptacle 310 may include a buffer solution therein. The buffer solution may include a pH-stabilizing agent and an ionic strength modifier. The kit 300 may further include beads 320 suitable for mechanical homogenization through bead beating. The kit 300 may further include a predefined quantity of lytic enzymes 330. The kit 300 may further include a predefined quantity of a chaotropic agent 340. The kit 300 may further include a predefined quantity of an adsorbent 350. The kit 300 may optionally include additional tools 360 for further mechanical disruption of the sample.

FIG. 3B illustrates an example kit 300 for extracting high molecular weight DNA 271 from a stool sample 272 containing prokaryotic cells 274. In some embodiments, the kit 300 includes a first container 362 that houses a buffer solution 364 used during initial preparation of the biological sample. The buffer solution 364 may include a pH-stabilizing agent 366, such as Tris-HCl, HEPES, MOPS, phosphate buffer systems, or combinations thereof. The pH-stabilizing agent 366 may assist in maintaining a controlled pH environment during handling of nucleic acids. The buffer solution 364 may also include an ionic strength modifier 367, such as sodium chloride, potassium acetate, sodium acetate, ammonium salts, or combinations thereof. The ionic strength modifier 367 may assist in maintaining ionic conditions that support enzymatic activity and nucleic acid stability. In some embodiments, the buffer solution 364 is provided in a premeasured liquid form, while in other embodiments it may be provided as a dry concentrate for reconstitution prior to use.

In some embodiments, the kit 300 further includes one or more beads 320 for mechanical homogenization through bead beating. The beads 320 may include glass 293 beads, ceramic 294 beads, zirconia beads, silica beads, or combinations thereof. The beads 320 may vary in diameter, such as from about 0.05 mm to about 3 mm, depending on the desired mechanical disruption intensity. Smaller beads may assist in disrupting bacterial aggregates, while larger beads may assist in breaking apart fibrous material present in stool matrices. In some embodiments, the beads 320 are selected to provide mechanical disruption of microbial cell structures without excessive fragmentation of nucleic acids. In some embodiments, bead composition may be selected based on durability or density to support repeated mixing cycles. The beads 320 may be supplied in sterile containers or preloaded within sample tubes.

In some embodiments, the kit 300 includes at least one receptacle 310 that contains a lytic enzyme 330. The lytic enzyme 330 may include lysozyme 283, mutanolysin 284, lysostaphin 285, or a combination 286. Lysozyme 283 may act on peptidoglycan structures present in many bacterial cell walls. Mutanolysin 284 may be used to disrupt specific Gram-positive organisms. Lysostaphin 285 may be used to target staphylococcal cell walls. In some embodiments, the lytic enzyme 330 may be provided in a lyophilized format for stability during storage. In some embodiments, multiple enzyme compositions may be provided to accommodate variation in microbial populations across stool samples.

In some embodiments, the kit 300 further includes a second container 399 that includes a chaotropic agent 368. The chaotropic agent 368 may include guanidinium thiocyanate, guanidinium hydrochloride, urea-based solutions, or similar protein-denaturing compounds. The chaotropic agent 368 may assist in disrupting protein structures and releasing nucleic acids from cellular components. In some embodiments, the chaotropic agent 368 may be provided as a liquid concentrate or as a solid composition for reconstitution. In some embodiments, the chaotropic agent 368 may be introduced after enzymatic treatment to support further separation of DNA from proteins and lipids. In some embodiments, the concentration of the chaotropic agent 368 may be adjusted depending on sample density or complexity. In some embodiments, the chaotropic agent 368 may assist in maintaining DNA solubility during downstream processing steps.

In some embodiments, the kit 300 further includes a third container 370 that includes an adsorbent 372. The adsorbent 372 may include polyvinylpolypyrrolidone, silica-based materials, activated carbon, or other contaminant-binding substances. The adsorbent 372 may bind and remove inhibitory compounds commonly present in stool samples. For example, humic substances, polysaccharides, and residual proteins may be captured by the adsorbent 372. In some embodiments, the adsorbent 372 may be provided as a particulate material. In some embodiments, the adsorbent 372 may be provided as a slurry or suspension. In some embodiments, the adsorbent 372 may assist in clarifying the solution prior to nucleic acid recovery.

In some embodiments, the components of the kit 300 may be used sequentially or otherwise in combination to support staged extraction of high molecular weight DNA 271. The buffer solution 364 may be used to suspend and stabilize the stool sample 272. Mechanical homogenization may be performed using the beads 320 to disrupt microbial cell structures. The lytic enzyme 330 may then be introduced to further degrade prokaryotic cell walls. The chaotropic agent 368 may subsequently be added to denature proteins and assist with nucleic acid release. The adsorbent 372 may then be introduced to bind contaminants prior to DNA recovery. These steps may be performed in sequence or modified depending on sample characteristics.

In some embodiments, the kit 300 may be packaged for single-use applications or for multiple extractions. The components may be stored in separate sterile containers to maintain stability prior to use. In some embodiments, the kit 300 may further include instructions for use, additional receptacles, and/or mixing vessels. In some embodiments, the kit 300 may be fielded for use in laboratory environments or field-based sample collection. In some embodiments, temperature-stable packaging may be used to protect enzyme activity during transport. In some embodiments, the kit 300 may include labeling and/or calibration markers to assist with consistent reagent addition.

FIG. 3C illustrates additional features of the kit 300 for extracting high molecular weight DNA 271 from a stool sample 272 containing prokaryotic cells 274. In some embodiments, the buffer solution provided in the kit 300 is applied to the stool sample 272 based on a determined weight 390 of the stool sample. The weight 390 may be measured prior to processing using a scale or other measuring device. In some embodiments, the volume of buffer solution introduced to the stool sample 272 may be selected to correspond to the measured weight of the sample. For example, larger stool samples may receive a greater volume of buffer solution to maintain a desired ratio between sample mass and liquid volume. In some embodiments, maintaining a selected ratio between buffer solution and sample mass may support uniform mixing and stabilization of nucleic acids during early processing steps. In some embodiments, the kit 300 may include markings, reference charts, or instructions 394 that indicate buffer volumes corresponding to different sample weights.

In some embodiments, an amount of an adsorbent included in the kit 300 may be applied to the stool sample 272 based on a predetermined binding capacity 392 of the adsorbent relative to the sample. The binding capacity 392 may correspond to the amount of contaminants expected to be present in stool samples of a given mass or composition. In some embodiments, the quantity of adsorbent introduced into the sample may be selected to correspond to the measured weight 390 of the stool sample. For example, a greater amount of adsorbent may be introduced when processing larger or more complex samples. In some embodiments, the kit 300 may include guidance within the instructions 394 indicating adsorbent quantities corresponding to sample weight ranges. In some embodiments, applying the adsorbent according to binding capacity 392 may assist in reducing inhibitory substances prior to nucleic acid recovery.

In some embodiments, the kit 300 may further include at least one additional tool for mechanical disruption of the stool sample 272 following enzymatic treatment. The additional tool may include a vortex adapter, bead mill accessory, ultrasonic probe, homogenizer, or other mechanical device. In some embodiments, the additional mechanical tool may be used after introduction of lytic enzymes to increase disruption of remaining microbial cell structures. In some embodiments, the additional tool may be used to assist with mixing of the chaotropic agent and adsorbent within the sample. In some embodiments, the additional tool may support processing of samples with higher microbial density or fibrous content. In some embodiments, the additional tool may be reusable or provided as a disposable component.

In some embodiments, the kit 300 may further include a temperature-controlled incubation unit 393 or instructions 394 for incubating the stool sample 272 with the buffer solution at a predefined temperature. The temperature-controlled incubation unit 393 may include a heat block, incubator, water bath, or other device capable of maintaining a selected temperature range. In some embodiments, incubation may occur during enzymatic treatment to support activity of the lytic enzyme. In some embodiments, incubation may also occur during chemical processing stages involving the chaotropic agent. In some embodiments, the instructions 394 may specify temperature ranges and time durations for different stages of the extraction process. In some embodiments, maintaining the stool sample 272 at a predefined temperature during incubation may support consistent processing across multiple samples.

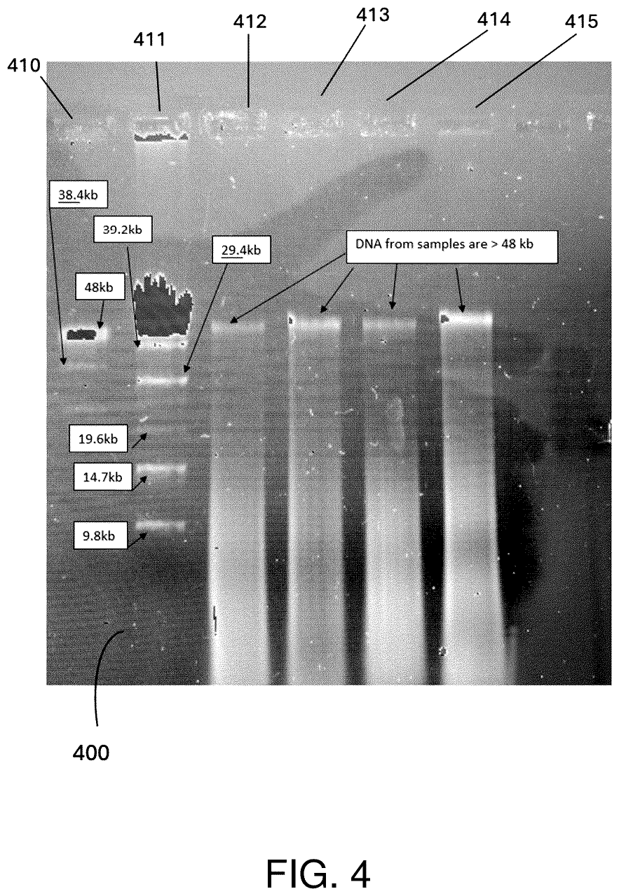

FIG. 4 shows an image 400 of agarose gel electrophoresis results of an initial extraction, with lanes containing 2 μL of the extracted DNA and a molecular weight marker in the leftmost lanes 410, 411. The first DNA size standard ladder used, shown in lane 410, is the CHEF DNA sized Standard 8-48 kb, which spans from 8.3 kb to 48.5 kb. The second ladder, the CHEF DNA Size Standard 5 kb shown in lane 411, includes fragments ranging from 4.9 kb to 98 kb. The additional lanes 412, 413, 414, and 415 represent four replicate samples, each showing DNA molecular weights around approximately 48.5 kb, confirming the successful extraction of high molecular weight DNA.

FIG. 4 demonstrates that the high molecular weight DNA samples 412, 413, 414, and 415 have a higher molecular weight than about 40 kb (e.g., each sample has a molecular weight of about 48 kb), as evidenced by their migration in the gel being above the largest band in the first CHEF DNA size standard ladder in lane 410, which is 48.5 kb.

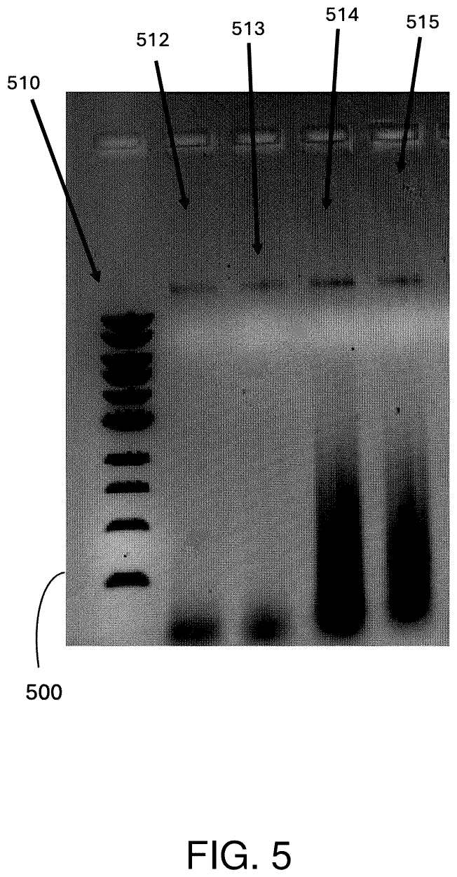

FIG. 5 shows an image 500 of high molecular weight DNA extraction after undergoing a phenol/chloroform clean-up step. Following the results 400 of the initial extraction shown in FIG. 4, the DNA samples were mixed with a volume of phenol/chloroform/isoamyl alcohol (25:24:1) to remove contaminants such as proteins and lipids. The mixture was centrifuged at about 12,000 rpm to about 16,000 rpm×g for about 3 minutes to about 7 minutes to separate the phases. The aqueous phase containing the DNA was collected. The collected aqueous phase was subjected to ethanol precipitation to recover the DNA. The DNA was then centrifuged at about 12,000 rpm to 16,000 rpm×g for about 5 minutes to about 15 minutes. The DNA was washed with 70% ethanol and then centrifuged for 10,000 rpm to 16,000 rpm×g for about 1 to about 3 minutes. The pellets were air-dried and re-suspended in a suitable buffer.

FIG. 5 shows an image 500 of agarose gel electrophoresis results of the phenol/chloroform cleaned DNA. The first lane 510 on the left is a 1 kb DNA ladder, which serves as a size reference. The subsequent lanes 512, 513, 514, and 515 represent the same four biological samples depicted in FIG. 4 in lanes 412, 413, 414, and 415 after being subjected to phenol/chloroform clean-up. The phenol/chloroform step may be used to remove residual contaminants to increase the purity of the high molecular weight DNA for downstream applications.

FIG. 5 demonstrates that the phenol/chloroform clean-up effectively purifies the DNA samples, as evidenced by the clear, distinct bands corresponding to high molecular weight DNA in lanes 512, 513, 514, and 515. These samples show minimal smearing and reduced degradation, indicating successful removal of proteins and other contaminants. The largest band in the 1 kb DNA ladder in lane 510 confirms that the high molecular weight DNA samples are above 10 kb, consistent with high molecular weight DNA suitable for further applications.

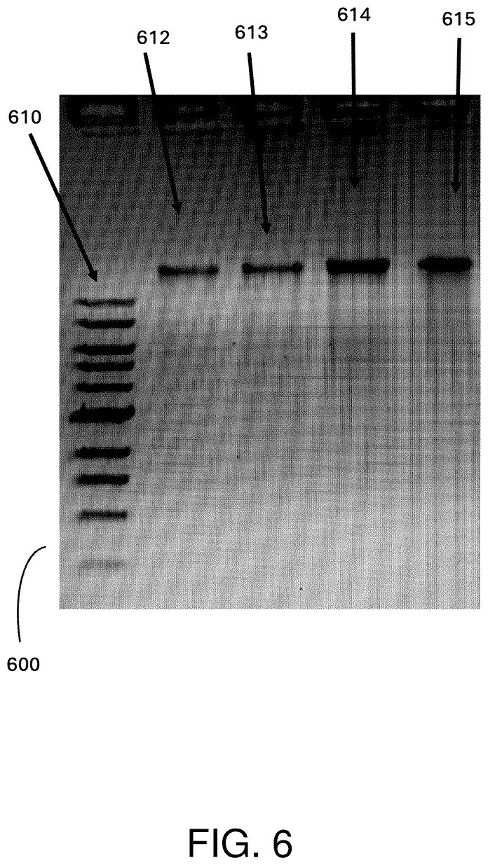

FIG. 6 shows an image 600 of high molecular weight DNA extraction after RNAse purification. Following the phenol/chloroform clean-up 500 depicted in FIG. 5, the DNA samples were treated with RNAse A to remove any residual RNA. The samples were incubated at about 37° C. for 30 minutes to allow for complete digestion of RNA contaminants.

After RNAse treatment, the DNA was purified using ethanol precipitation. The DNA was washed with 70% ethanol, dried, and re-suspended in a suitable buffer. FIG. 6 shows the agarose gel electrophoresis results 600 of the RNAse-treated DNA. The first lane 610 on the left is a 1 kb DNA ladder, which provides a size reference. The subsequent lanes 612, 613, 614, and 615 display the same four biological samples depicted in both FIG. 4 lanes 412, 413, 414, and 415 and FIG. 5 lanes 512, 513, 514, and 515 after undergoing RNAse treatment and ethanol precipitation. The results confirm the successful removal of RNA contaminants, resulting in high-purity DNA suitable for advanced genomic analyses.

FIG. 6 further confirms the integrity and purity of the DNA samples after RNAse treatment. The distinct bands in lanes 612, 613, 614, and 615 representing the high molecular weight DNA samples, compared against the 1 kb DNA ladder in lane 610, indicate the absence of RNA contamination. The DNA fragments in these samples remain high molecular weight, with minimal smearing and degradation, demonstrating the effectiveness of the RNAse purification step.

### Working Examples

Working Example 1. High Molecular Weight DNA Extraction from Human Stool Samples

Stool samples were collected from human subjects and stored at 4° C. until processing. A prechilled buffer solution containing about 50 mM Tris-HCl and about 1 mM EDTA to inhibit nuclease activity was added to the stool samples. The prepared samples underwent a first mechanical lysis step using vortexing for 1.5 minutes to partially disrupt the cell walls and release some cellular contents. Following the initial vortexing, the samples were centrifuged at about 14,000 rpm for 3 minutes. The supernatant was transferred to a clean microcentrifuge tube.

The samples were treated with a combination of lytic enzymes. The lysozyme was used at a concentration of about 230 mg/mL, the mutanolysin was used at a concentration of about 24 KU/mL, and the lysostaphin was used at a concentration of about 3 KU/mL. The samples were incubated at about 37° C. for 45 minutes to ensure thorough enzymatic lysis of the prokaryotic cell walls. After enzymatic treatment, the samples were further processed with about 250 μL of 5 M guanidinium thiocyanate and mixed gently for 50 seconds. This was followed by the addition of about 300 μL of 12% N-Laurylsarcosine, mixing well, and performing a quick spin down. The mixture was then incubated at about 42° C. for 5 minutes with mixing at 350 rpm, followed by an incubation at 65° C. for 75 minutes to denature proteins and other contaminants.

Next, about 10 mg of PVPP was added to the treated mixture to bind and remove contaminants such as humic substances and polysaccharides. The sample was mixed well and subjected to a quick spin down. The sample was then centrifuged at about 15,000×g for about 4 minutes to facilitate the separation of PVPP-bound contaminants from the nucleic acids. The supernatant containing the high molecular weight DNA was carefully transferred to a new 2 mL centrifuge tube for further processing or analysis.

Working Example 2. High Molecular Weight DNA Extraction from Nonhuman Primate Stool Samples

Stool samples from nonhuman primates (e.g., macaques) were collected and processed using the optimized methods. A prechilled buffer solution containing about 50 mM Tris-HCl and about 1 mM EDTA was added to the stool samples. The prepared samples underwent mechanical lysis using bead beating with glass beads (0.1 mm diameter) for 2 minutes. Following the initial mechanical lysis, the samples were centrifuged at 15,000 rpm for about 3 minutes. The supernatant was then transferred to a clean microcentrifuge tube.

The samples were then treated with a combination of lytic enzymes. The lysozyme was used at a concentration of about 260 mg/mL, mutanolysin at a concentration of about 25 KU/mL, and lysostaphin at a concentration of about 4 KU/mL, and incubated at about 37° C. for 45 minutes. After enzymatic treatment, the samples were further processed by adding about 250 μL of 5 M guanidinium thiocyanate and mixing gently for about 45 seconds. This was followed by the addition of about 300 μL of 10% N-Laurylsarcosine, mixing well, and performing a quick spin down. The mixture was then incubated at 37° C. for 10 minutes with mixing at 250 rpm, followed by an incubation at about 70° C. for about 60 minutes to denature proteins and other contaminants.

Next, about 20 mg of PVPP was added to the treated mixture for contaminant removal. The sample was mixed well and subjected to a quick spin down. The sample was then centrifuged at about 20,000×g for about 4 minutes to facilitate the separation of PVPP-bound contaminants from the nucleic acids. The supernatant containing the high molecular weight DNA was carefully transferred to a new 2 mL centrifuge tube for further processing or analysis.

Working Example 3. High Molecular Weight DNA Extraction from Mouse Stool Samples

Stool samples from mice were collected and stored at 4° C. until processing. A prechilled buffer solution containing about 50 mM Tris-HCl and about 1 mM EDTA was added to the stool samples. The prepared samples underwent mechanical lysis using bead beating with ceramic beads (0.5 mm diameter) for about 2 minutes. Following the initial mechanical lysis, the samples were centrifuged at about 14,000 rpm for about 3 minutes. The supernatant was then transferred to a clean microcentrifuge tube.

The samples were treated with a combination of lytic enzymes. The lysozyme was used at a concentration of about 250 mg/mL, mutanolysin at a concentration of about 28 KU/mL, and lysostaphin at a concentration of about 5 KU/mL. The samples were incubated at about 37° C. for about 30 minutes. After enzymatic treatment, the samples were further processed by adding about 250 μL of 4 M guanidinium thiocyanate and mixing gently for about 45 seconds. This was followed by the addition of about 300 μL of 10% N-Laurylsarcosine, mixing well, and performing a quick spin down. The mixture was incubated at about 37° C. for about 10 minutes with mixing at about 300 rpm, followed by an incubation at about 70° C. for about 1 hour to denature proteins and other contaminants.

Next, about 15 mg of PVPP was added to remove contaminants, followed by phenol/chloroform extraction and ethanol precipitation. The sample was mixed well and subjected to a quick spin down. The sample was then centrifuged at about 20,000×g for about 4 minutes to facilitate the separation of PVPP-bound contaminants from the nucleic acids.

### Prophetic Examples

### Prophetic Example 1

A patient presenting with chronic abdominal pain and irregular bowel movements is suspected of having colorectal cancer. A stool sample is processed using the methods described herein to extract high molecular weight DNA. Sequencing reveals an overrepresentation of Fusobacterium nucleatum, a bacterium linked to colorectal cancer. Subsequent colonoscopy confirms early-stage cancer, enabling timely intervention and personalized treatment.

### Prophetic Example 2

Stool samples are collected from patients with Type 2 diabetes and healthy controls. Using the methods described herein, high molecular weight DNA is extracted and sequenced, which identifies a reduction (e.g., below a predefined threshold) in beneficial bacteria such as Akkermansia muciniphila in diabetic patients. A probiotic supplement including A. muciniphila is administered to a patient having or at risk of having Type 2 diabetes to improve insulin sensitivity and glycemic control.

### Prophetic Example 3

DNA is extracted from stool samples from an animal dietary model. The high-quality, extracted DNA is sequenced. The sequencing data are analyzed to determine the impact of one or more diets on microbial diversity and/or function.

### Prophetic Example 4

A patient with irritable bowel syndrome (IBS) undergoes microbiome profiling using the methods described herein. Genomic and sequencing analyses identify specific bacterial imbalances contributing to the patient's symptoms. Based on these results, a personalized diet plan is determined that eliminates foods that feed harmful bacteria while promoting beneficial microbes. The patient experiences one or more improvements in IBS symptoms.

### Prophetic Example 5

Stool samples may be collected from fish and other aquatic animals for environmental impact analyses, for example, due to agricultural runoff into river ecosystems. Using the methods described herein, DNA is extracted and analyzed to identify surrounding agricultural areas having higher levels of bacteria associated with nutrient pollution. These findings may be used to determine or recommend improved agricultural practices to protect river ecosystems.

### Examples

Embodiment 1. A method for extracting high molecular weight DNA from prokaryotic cells in stool samples, the method comprising: receiving a biological sample comprising prokaryotes; preparing the biological sample in a prechilled buffer solution; homogenizing the biological sample through a first mechanical means for partial lysis; partially disrupting prokaryotic cell walls by treating the biological sample with at least one lytic enzyme to release genetic information; treating the biological sample with a chaotropic agent; disrupting the biological sample by a second mechanical means; and applying an adsorbent to the biological sample.
Embodiment 2. The method of embodiment 1, wherein the prechilled buffer solution comprises a pH-stabilizing agent and an ionic strength modifier.
Embodiment 3. The method of embodiment 1, wherein the first mechanical means to homogenize the biological sample comprises vortexing the biological sample for a predefined period of time.
Embodiment 4. The method of embodiment 1, wherein the first mechanical means comprises bead beating.
Embodiment 5. The method of embodiment 4, wherein the bead beating is performed using beads of a predefined diameter.
Embodiment 6. The method of embodiment 1, wherein the at least one lytic enzyme is lysozyme, mutanolysin, lysostaphin, or a combination thereof.
Embodiment 7. The method of embodiment 1, wherein the chaotropic agent comprises guanidinium thiocyanate.
Embodiment 8. The method of embodiment 7, further comprising determining a prokaryotic cell density of the biological sample; and adjusting a concentration of the chaotropic agent based on the prokaryotic cell density in the biological sample.
Embodiment 9. The method of embodiment 1, further comprising incubating the biological sample with the prechilled buffer solution at a predefined temperature to increase enzyme activity before mechanical homogenization.
Embodiment 10. The method of embodiment 9, wherein the predefined temperature for incubation is based on a predetermined activity of the at least one lytic enzyme.
Embodiment 11. The method of embodiment 1, further comprising centrifuging the biological sample to separate one or more adsorbent-bound contaminants from a nucleic acid in the biological sample.
Embodiment 12. The method of embodiment 1, further comprising removing at least one enzymatic inhibitor prior to adding the chaotropic agent.
Embodiment 13. A reagent system for extracting high molecular weight DNA from a stool sample comprising one or more prokaryotic cells, the reagent system comprising: a buffer solution for preparing a biological sample, wherein the buffer solution comprises a pH-stabilizing agent and an ionic strength modifier; at least one mechanical homogenization tool configured for bead beating with one or more beads; at least one lytic enzyme for disrupting prokaryotic cell walls, the at least one lytic enzyme comprising lysozyme, mutanolysin, lysostaphin, or a combination thereof; a chaotropic agent configured for treating the biological sample; and an adsorbent configured for contaminant removal.
Embodiment 14. The reagent system of embodiment 13, wherein the chaotropic agent comprises guanidinium thiocyanate.
Embodiment 15. The reagent system of embodiment 13, wherein the buffer solution further comprises ethylenediaminetetraacetic acid (EDTA) configured to inhibit nuclease activity.
Embodiment 16. The reagent system of embodiment 13, wherein the buffer solution is part of a modular buffer system comprising a series of buffers.
Embodiment 17. The reagent system of embodiment 16, wherein each buffer in the series of buffers corresponds to a different stage of the high molecular weight DNA extraction.
Embodiment 18. The reagent system of embodiment 13, further comprising: one or more mechanical disruption tools; and one or more materials configured for incubating the biological sample with the buffer solution at a predefined temperature.
Embodiment 19. The reagent system of embodiment 13, wherein the one or more beads comprise a glass or a ceramic material.
Embodiment 20. A kit for extracting high molecular weight DNA from stool samples comprising prokaryotic cells, the kit comprising: a first container comprising a buffer solution comprising a pH-stabilizing agent and an ionic strength modifier; one or more beads configured for mechanical homogenization through bead beating, wherein the one or more beads comprise glass or ceramic; at least one receptacle comprising at least one lytic enzyme selected from a list consisting of: lysozyme, mutanolysin, lysostaphin, or a combination thereof; a second container comprising a chaotropic agent; and a third container comprising an adsorbent configured for binding and removing one or more contaminants from a stool sample.
Embodiment 21. The kit of embodiment 20, wherein the buffer solution is configured to be applied to the stool sample based on a determined weight of the stool sample.
Embodiment 22. The kit of embodiment 20, wherein an amount of the adsorbent is configured to be applied to the stool sample based on a predetermined binding capacity of the adsorbent for the stool sample.
Embodiment 23. The kit of embodiment 20, further comprising at least one additional tool for mechanical disruption of the stool sample following enzymatic treatment.
Embodiment 24. The kit of embodiment 20, further comprising a temperature-controlled incubation unit or instructions for incubating the stool sample with the buffer solution at a predefined temperature.

As used in the description and claims, the singular form “a”, “an”, and “the” include both singular and plural references unless the context clearly dictates otherwise. For example, the term “sample” may include, and is contemplated to include, a plurality of samples. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

What is claimed is:

1. A method for extracting high molecular weight DNA from prokaryotic cells in stool samples, the method comprising:
receiving a biological sample comprising prokaryotes;
preparing the biological sample in a prechilled buffer solution;
homogenizing the biological sample through a first mechanical means for partial lysis;
partially disrupting prokaryotic cell walls by treating the biological sample with at least one lytic enzyme to release genetic information;
treating the biological sample with a chaotropic agent;
disrupting the biological sample by a second mechanical means; and
applying an adsorbent to the biological sample.

2. The method of claim 1, wherein the prechilled buffer solution comprises a pH stabilizing agent and an ionic strength modifier.

3. The method of claim 1, wherein the first mechanical means to homogenize the biological sample comprises vortexing the biological sample for a predefined period of time.

4. The method of claim 1, wherein the first mechanical means comprises bead beating.

5. The method of claim 4, wherein the bead beating is performed using beads of a predefined diameter.

6. The method of claim 1, wherein the at least one lytic enzyme is lysozyme, mutanolysin, lysostaphin, or a combination thereof.

7. The method of claim 1, wherein the chaotropic agent comprises guanidinium thiocyanate.

8. The method of claim 7, further comprising determining a prokaryotic cell density of the biological sample; and adjusting a concentration of the chaotropic agent based on the prokaryotic cell density in the biological sample.

9. The method of claim 1, further comprising incubating the biological sample with the prechilled buffer solution at a predefined temperature to increase enzyme activity before mechanical homogenization.

10. The method of claim 9, wherein the predefined temperature for incubation is based on a predetermined activity of the at least one lytic enzyme.

11. The method of claim 1, further comprising centrifuging the biological sample to separate one or more adsorbent-bound contaminants from a nucleic acid in the biological sample.

12. The method of claim 1, further comprising removing at least one enzymatic inhibitor prior to adding the chaotropic agent.

13. A reagent system for extracting high molecular weight DNA from a stool sample comprising one or more prokaryotic cells, the reagent system comprising:
a buffer solution for preparing a biological sample, wherein the buffer solution comprises a pH-stabilizing agent and an ionic strength modifier;
at least one mechanical homogenization tool configured for bead beating with one or more beads;
at least one lytic enzyme for disrupting prokaryotic cell walls, the at least one lytic enzyme comprising lysozyme, mutanolysin, lysostaphin, or a combination thereof;
a chaotropic agent configured for treating the biological sample; and
an adsorbent configured for contaminant removal.

14. The reagent system of claim 13, wherein the chaotropic agent comprises guanidinium thiocyanate.

15. The reagent system of claim 13, wherein the buffer solution further comprises ethylenediaminetetraacetic acid (EDTA) configured to inhibit nuclease activity.

16. The reagent system of claim 13, wherein the buffer solution is part of a modular buffer system comprising a series of buffers.

17. The reagent system of claim 16, wherein each buffer in the series of buffers corresponds to a different stage of the high molecular weight DNA extraction.

18. The reagent system of claim 13, further comprising:
one or more mechanical disruption tools; and
one or more materials configured for incubating the biological sample with the buffer solution at a predefined temperature.

19. The reagent system of claim 13, wherein the one or more beads comprise a glass or a ceramic material.

20. A kit for extracting high molecular weight DNA from stool samples comprising prokaryotic cells, the kit comprising:
a first container comprising a buffer solution comprising a pH-stabilizing agent and an ionic strength modifier;
one or more beads configured for mechanical homogenization through bead beating, wherein the one or more beads comprise glass or ceramic;
at least one receptacle comprising at least one lytic enzyme selected from a list consisting of:
lysozyme, mutanolysin, lysostaphin, or a combination thereof;
a second container comprising a chaotropic agent; and
a third container comprising an adsorbent configured for binding and removing one or more contaminants from a stool sample.

21. The kit of claim 20, wherein the buffer solution is configured to be applied to the stool sample based on a determined weight of the stool sample.

22. The kit of claim 20, wherein an amount of the adsorbent is configured to be applied to the stool sample based on a predetermined binding capacity of the adsorbent for the stool sample.

23. The kit of claim 20, further comprising at least one additional tool for mechanical disruption of the stool sample following enzymatic treatment.

24. The kit of claim 20, further comprising a temperature-controlled incubation unit or instructions for incubating the stool sample with the buffer solution at a predefined temperature.
